(19) United States
(12) Patent Application Publication
(10) Pub. No.: US 2004/0092018 A1
(43) Pub. Date: May 13, 2004

(54) Insertional mutagenesis technique

(75) Inventors: Charalambos Savakis, Heraklion (GR); Frank Grosveld, Rotterdam (NL)

Correspondence Address: PALMER & DODGE, LLP, KATHLEEN M. WILLIAMS, 111 HUNTINGTON AVENUE, BOSTON, MA, 02199, US

(73) Assignee: Minos Biosystems Limited

(21) Appl. No.: 10/634,314

(22) Filed: Aug. 5, 2003

Related U.S. Application Data
(63) Continuation of application No. PCT/GB02/00484, filed on Feb. 5, 2002.

(30) Foreign Application Priority Data
Feb. 5, 2001 (GB) 0102816.6
Mar. 7, 2001 (GB) 0105642.3

(51) Int. Cl. C12N 15/86; A01K 67/00

(52) U.S. Cl. 435/456; 800/8

(57) ABSTRACT

The inventon provides a method for producing a library of genetic mutations in a cell population by insertional mutagenesis, wherein a viral vector comprising a transposon is used to deliver said transposon to said cell population, which cell population stably expresses the cognate transposase for said transposon, and the transposon is mobilised to give a rise to the genetic mutations.

The present invention relates to a method for targeting genes in a cell using a combination of integrating vectors. Such vectors may be viruses and transposons. The method according to the invention comprises the stable provision of a transposase activity, to catalyse transposon mobilisation, in the cell. The techniques described herein are generally useful for genetic research in whole organisms, including animals, for example mammals, including humans, insects, and cells, primary cell cultures and cell lines derived therefrom, and in particular for functional analysis of mammalian genomes.

The introduction of exogenous DNA into the genome is a critical step for the study of molecular genetics. For example, insertion events involving viruses or homologous recombination of DNA are known, and may be used to give rise to novel phenotypic variations in the cells, which can be traced back to insertion events in the cell genome and hence the sequences or genes responsible for the phenotype when not normally active. Insertions may have small phenotypic effects, for example resulting from the insertion of a few amino acids into the sequence of a polypeptide or decreased expression of the gene. Alternatively, the effects may be more pronounced, possibly including the complete inactivation of a gene.

Insertion events may be detected by screening for the presence of the vector, by probing for the nucleic acid sequence thereof.

Moreover, insertion vectors may be used to upregulate the expression of genes. For example, a vector may be modified to Include an enhancer or other transcriptional activation element. Insertion of such a transposon in the vicinity of a gene upregulates expression of the gene or gene locus. This embodiment has particular advantage in the isolation of oncogenes, which may be identified in transformed cells by localisation of the vector.

Transposons are genetic elements which are capable of “jumping” or transposing from one position to another within the genome of a species. They are widely distributed amongst animals, including insects.

Transposons are active within their host species due to the activity of a transposase protein encoded by the elements themselves. Advances in the understanding of the mechanisms of transposition have resulted in the development of genetic tools based on transposons which can be used for gene transfer.

Members of the Tc1/mariner family have terminal inverted repeats which end with a highly conserved sequence (CAGTGC). They integrate into the sequence TA and contain a single gene encoding a related polypeptide. An alignment of the open reading frames found in the Tc1-like elements has been published by Henikoff (1992) New Biologist 4, 382-388. Other Tc1/mariner elements have been detected by hybridisation, PCR amplification or database searches in different nematode species (Abad et al., (1991) J. Mol. Evol. 33, 251-258; Sedensky et al., (1994) Nucleic Acids Res. 22, 1719-1723, planarians (Garcia-Fernandez et al., (1993) Nature 364, 109, arthropods (Bigot et al., (1994) Proc. Natl. Acad. Sci. USA 91, 3408-3412). and vertebrates (Henikoff, (1992) New Biologist 4, 382-388; Goodier and Davidson, (1994) J. Mol. Biol. 241, 26-34). More distantly related members of the Tc1/mariner family have been found in bacteria and ciliated protozoa.

Minos is a transposable element of the Tc1 superfamily derived from Drosophila (Franz and Savakis, (1991) NAR 19:6646). It is described in U.S. Pat. No. 5,840,865, which is incorporated herein by reference in its entirety. The use of Minos to transform insects is described in the foregoing U.S. patent.

Mariner is a transposon originally isolated from Drosophila, but since discovered in several invertebrate and vertebrate species. The use of mariner to transform organisms is described in International patent application WO99/09817.

Hermes is derived from the common housefly. Its use in creating transgenic insects is described in U.S. Pat. No. 5,614,398, incorporated herein by reference in its entirety.

PiggyBac is a transposon derived from the baculovirus host Trichplusia ni. Its use for germ-line transformation of Medfly has been described by Handler et al., (1998) PNAS (USA) 95:7520-5.

European Patent Application 0955364 (Savakis et al., the disclosure of which is incorporated herein by reference) describes the use of Minos to transform cells, plants and animals. The generation of transgenic mice comprising one or more Minos insertions is described.

International Patent Application WO99/07871 describes the use of the Tc1 transposon from C. elegans for the transformation of C. elegans and a human cell line.

The use of Drosophila P-elements in D. melanogaster for enhancer trapping and gene tagging has been described; see Wilson et al., (1989) Genes dev. 3:1301; Spradling et al., (1999) Genetics 153:135. Our copending UK patent applications 0020843.9 and 0006753.8 describe the use of transposons in the genomic analysis of cells and transgenic animals respectively.

However, a number of disadvantages are associated with current technologies useful for insertional mutagenesis of the genome. Firstly, neither viruses, naked DNA nor transposons integrate completely at random in the genome.

For retroviral DNA insertions it is known that the integration sites are near hypersensitive sites in the host genome (such as DNase 1 hypersensitive sites, and often near transcribed genes) which limits the randomness of insertions. Integration is in principle a recombination process using short homologies between the incoming DNA and the insertion site. Hence there will be a difference between the likelihood of integration at different sites dependent or their accessibility. The latter depends on the state of the chromatin at any site, but also on the type of recombination and the homology in question. Different enzymes are responsible for different integrations: viral integration is controlled by a viral integrase, whilst transposons depend on a cognate transposase.

In a library of insertions it is desirable to hit as many genes as possible and for any given gene to knock it out completely or at least downregulate its expression substantially. However, using known approaches, in any given cell only one of the two alleles will be hit. The opposite, upregulation, is easier as it is possible to integrate a strong transcriptional enhancer near one allele of a given gene. The other allele does not have to be upregulated to achieve the desired effect.

Although transposon insertion is more random than viral or homologous insertion, it suffers from inefficiencies in transposon mobilisation. For example, in WO99/07871 the use of transposons in mammalian cells, driven by transposase provided, for xample, on DNA vectors is described. However, this approach is not demonstrated; in fact, use of DNA vectors to deliver a transposase gene is highly inefficient and transposition cannot reliably be achieved. In WO99107871 transposon mobilisation is only demonstrated where the transposase protein is supplied exogenously to the cell.

Finally, perhaps the most serious bottleneck in developing efficient transposable element based mutagenesis methods is introducing transposon DNA into cells. Several methods for introducing plasmid DNA into cultured cells exist, including calcium coprecipitation, lipofection, electroporation and direct injection; results vary considerably with cell line and method.

### SUMMARY OF THE INVENTION

We have now developed a technique which overcomes the problems of the prior art. In the present invention, mobilisation of transposons is achieved efficiently; limitations resulting from non-randomness of integration are reduced; and genes may be efficiently disrupted using only single-allele insertional events.

According to a first aspect, therefore, there is provided a method for producing a library of genetic mutations in a cell population by insertional mutagenesis, wherein a composite vector comprising at least two nucleic acid elements capable of insertion into the cell genome by different mechanisms is used to give rise to two or more mechanistically different insertional events in said cell population.

For example, a viral vector comprising a transposable element may be used to effect both viral integration and transposon mobilisation in the cell population, exploiting the ability of the viral and transposon components of the invention to integrate into different parts of the genome with differing frequency.

In a second aspect the invention provides a method for producing a library of genetic mutations in a cell population by insertional mutagenesis, wherein a transposon is introduced into the population of cells, which population of cells stably expresses the cognate transposase for said transposon, and the transposon is mobilised to give rise to the genetic mutations.

It has been observed that the provision of a transposase gene in stable form in a cell achieves highly efficient transposon mobilisation, whilst transfection of the cell with transiently-expressed transposase, for example as encoded on a viral vector or plasmid, is inefficient.

The transposon is preferably delivered using a viral vector.

In a third aspect, the invention provides a method for producing a library of genetic mutations in a cell population by insertional mutagenesis in which insertion of a vector into a gene leads to gene inactivation, wherein the vector comprises an inducible promoter 5′ to the insertion site which drives the expression of an antisense transcript of said gene.

In the foregoing manner, both the alleles of the gene may be inactivated; one by insertional deletion, and the second by antisense RNA transcribed from the first allele which contains the inserted vector. The inducible promoter is advantageously a tetracycline promoter (Gossen, M., Freundlieb, S., Bender, G., Müiller, G., Hillen, W. and Bujard, H. (1995) Transcriptional activation by tetracycline in mammalian cells. Science 268, 1766-1769).

Advantageously, the vector is a viral vector which encodes one or more transposons. The cognate transposase(s) is or are advantageously stably expressed in the cell population.

In the broader context of the foregoing aspects of the invention, delivery of the nucleic acids may be accomplished by any available technique, including transformation/transfection, delivery by viral or non-viral vectors and microinjection. Each of these techniques is known in the art. Ribonucleic acids, in particular, may be delivered by microinjection or by viral transduction, particularly by RNA viruses.

In a preferred aspect of the invention, there is provided a method for producing a library of genetic mutations in a cell population by insertional mutagenesis, wherein a viral vector comprising a transposon is used to deliver said transposon to said cell population, which cell population stably expresses the cognate transposase for said transposon, and the transposon is mobilised to give rise to the genetic mutations.

It has been found that viral delivery of a transposon to a cell which stably expresses the cognate transposase gives highly efficient transposon mobilisation. For example, a genetically marked transposon is engineered into the genome of a virus that cannot replicate in the target cells. The virus is packaged, purified, and viral particles are used to infect the target cells. After infection, uncoating and (for RNA viruses) reverse transcription, the transposon DNA is available in the target cell for transposase-mediated transposition into chromosomal sites.

The virus may be an integrating or non-integrating virus. Where the virus is an integrating virus, the invention moreover provides the advantage of the first aspect thereof, namely, the provision of two mechanistically separate integrating elements in a single composite vector.

### BRIEF DESCRIPTION OF THE FIGURES

FIG. 1 shows the transposon MiLRgeo, inserted into the first intron of a hypothetical target gene. L and R are the inverted repeats of the Minos transposon.

FIG. 2 shows generation of a recombinant baculovirus vector carrying a transposon by homologous recombination.

FIG. 3 shows the BacMiSV40neo transposon virus. pA is the SV40 polyadenylation region and hyd are the Drosophila hydei genomic sequences flanking the original Minos transposable elements.

FIG. 4 shows the BacCMV/ILMi helper virus. pA is the polyadenylation site of the bovine growth hormone gene

FIG. 5 shows the pBI-L/ILMi helper plasmid. pA are polyadenylation sites. ILMi is the intronless Minos transposase gene.

FIG. 6 shows the PBO-MG1 lentiviral vector construct

FIG. 7 shows a Southern blot of genomic DNA from clones of MEL cells carrying an integrated copy of the lenti-Minos-GFP virus. The DNA was digested with BspE I and probed with a 3′LTR end fragment probe. Lanes 2 & 4 have DNA from the clones transfected with the plasmid pNT-1, carrying the CMV driven transposase gene resulting in a transposition that gives a new band that hybridises with end fragment probe.

### DETAILED DESCRIPTION OF THE INVENTION

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4th Ed, John Wiley & Sons, Inc (as well as the complete version Current Protocols in Molecule Biology).

Definitions

A “cell population” is a population of a suitable cell type in which it is desired to introduce genetic mutations. Suitable cell types are described below. The population is advantageously large in size, and may number anything up to 10¹⁵ or more. Advantageously, it is larger than 100 cells, and preferably larger than 1000 cells, for example 10⁴, 10⁵, 10⁶, 10⁷, 10⁸, 10⁹ cells or more. A cell population may moreover be a non-human animal, preferably a mammal or insect.

A “composite vector” is a nucleic acid vector capable of integrating into the genome of a cell which comprises two integrating elements. In this context, an “element” is a nucleic acid sequence which is capable of integrating into the genome of a cell.

“Mechanistically different” insertions are insertions which take place by different mechanisms—for example viral integration, transposon integration, homologous recombination and the like. Preferably, the integration events are catalysed by enzymes. Advantageously, mechanistically different insertional events are catalysed by different enzymes.

### PREFERRED ASPECTS OF THE INVENTION

Nucleic Acids

A nucleic acid, as referred to herein, may be any nucleic acid, including DNA and RNA, as well as synthetic nucleic acid homologues such as backbone-modified nucleic acids including methylphosphonates, phosphorothioates and phosphorodithioates, where both of the non-bridging oxygens are substituted with sulphur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire phosphodiester backbone with a peptide linkage.

Sugar modifications are also used to enhance stability and affinity. The α-anomer of deoxyribose may be used, where the base is inverted with respect to the natural β-anomer. The 2′-OH of the ribose sugar may be altered to form 2-O-methyl or 2′-O-allyl sugars, which provides resistance to degradation without comprising affinity.

Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

A ribonucleic acid, as referred to herein, may be natural or modified RNA. Advantageously, the RNA may comprise one or more of the modifications identified above.

Cells

The cell population may be any suitable cell type, including plant, insect and mammalian cells. The cells may be part of an organism, in primary culture, or established cell lines. Mammalian cells including (embryonic) stem cells are preferred. The method of the present invention may be used in transgenic organisms, such as transgenic insects, mammals or plants.

In general, cells for use in the methods of the invention may be derived from any source, such as prokaryote, yeast, plant and other higher eukaryote cells. Suitable prokaryotes include eubacteria, such as Gram-negative or Gram-positive organisms, such as E. coli, e.g. E. coli K-12 strains, DH5α and HB101, or Bacilli. Further host cells include eukaryotic microbes such as filamentous fungi or yeast, e.g. Saccharomyces cerevisiae. Higher eukaryotic cells include insect and vertebrate cells, particularly mammalian cells, including human cells, or nucleated cells from other multicellular organisms. The propagation of vertebrate cells in culture (tissue culture) is a routine procedure. Examples of useful mammalian host cell lines are epithelial or fibroblastic cell lines such as mouse embryonic stem (ES) cells, Chinese hamster ovary (CHO) cells, NIH 3T3 cells, HeLa cells or 293T cells.

Animal cells include cell lines derived from animals of the phyla cnidaria, ctenophora, platyhelminthes, nematoda, annelida, mollusca, chelicerata, uniramia, crustacea and chordata. Uniramians include the subphylum hexapoda that includes insects such as the winged insects. Chordates includes vertebrate groups such as mammals, birds, reptiles and amphibians. Particular examples of mammals include humans, non-human primates, cats, dogs, ungulates such as cows, goats, pigs, sheep and horses and rodents such as mice, rats, gerbils and hamsters.

Plant cells may be derived from plants including the seed-bearing plants angiosperms and conifers. Angiosperms include dicotyledons and monocotyledons. Examples of dicotyledonous plants include tobacco, (Nicotiana plumbaginifolia and Nicotiana tabacum), arabidopsis (Arabidopsis thaliana), Brassica napus, Brassica nigra, Datura innoxia, Vicia narbonensis, Vicia faba, pea (Pisum sativum), cauliflower, carnation and lentil (Lens culinaris). Examples of monocotyledonous plants include cereals such as wheat, barley, oats and maize.

Transposons

Any transposon may be used in the method of the invention. Preferably, the transposon is selected from the group consisting of Minos, mariner, Hermes and piggyBac. Advantageously, the transposon is Minos. Each transposon is advantageously employed with its natural cognate transposase, although the use of modified and/or improved transposases is envisaged.

The transposon preferably comprises a nucleic acid sequence encoding a heterologous polypeptide. This sequence will be integrated, together with the transposon, into the genome of the cell on transposon integration. Moreover, it will be excised, together with the transposon, when the latter excises on remobilisation. In a preferred embodiment, the heterologous polypeptide is a selectable marker. This allows cells having integrated transposons to be identified and the site of integration to be accurately mapped.

Transformation efficiency, expressed as percentage of individuals giving transformed progeny, is a crucial parameter in designing strategies for transgenesis, especially for species that are difficult to breed. Mobile element mediated transgenesis is usually based on two components; a transposon and the homologous or cognate transposase. For mobile elements of the Tc1 /mariner family, the presence of these two components during early embryogenesis is considered to be necessary and sufficient for integration of the transposon into host chromosomes, since transposases of Tc1, Mos1 and Himar1 can also catalyse transposition in vitro (Tosi, L. R. and Beverley, S. M. (2000) Nucleic Acids Res, 28, 784-90; Lampe, D. J., Churchill, M. E. and Robertson, H. M. (1996) Embo J, 15, 5470-9; and Franz, G. and Savakis, C. (1991) Nucleic Acids Res, 19, 6646). However, there is evidence that transpositional activity may not be proportional to the amount of transposase present; high concentrations of transposase may inhibit transposition in vitro (Lampe, D. J., Grant, T. E. and Robertson, H. M. (1998) Genetics, 149, 179-87) and in vivo (Loukeris, T. G., Arca, B., Livadaras, I., Dialektaki, G. and Savakis, C. (1995) Proc Natl Acad Sci U S A, 92, 9485-9; our own results). This work shows that the use of in vitro synthesised Minos transposase mRNA can result in high transformation efficiencies in both species that were tested, D. melanogaster, and the medfly C. capitata.

A transformation frequency of 3.2% has been accomplished in Drosophila melanogaster by injecting pMiw1, a non-autonomous Minos transposon marked with a wild-type version of the white gene, to pre-blastoderm embryos carrying a chromosomal source of transposase (Loukeris et al., Op. Cit.). Similar transformation frequencies (ca 1-6%) have been reported for Minos-mediated transformation of Drosophila, using the same transposon combined with a transposase expressing (helper) plasmid (Loukeris et al., Op. Cit.). The efficiency of transformation in these cases depends on a) the levels of transposase in germ line nuclei and b) the transformation procedure itself. Transposase levels may vary according to the promoter that drives its expression and, in the latter case, the amount of plasmid injected. Gradual improvements of technique have resulted in increased transformation efficiencies. In our hands, Minos-mediated transformation efficiency of up to 10% has been achieved in Drosophila (unpublished data), using various transposons and the helper plasmid that was originally used by Loukeris et al. (Loukeris et al., Op. Cit.).

Transformation rates of different insect species may vary widely, depending on the species and the transformation system. For example, transformation rates of 1% and 3-5% have been reported for Medfly with Minos and with piggyBac, respectively (Loukeris, T. G., Livadaras, I., Arca, B., Zabalou, S. and Savakis, C. (1995) Science, 270, 2002-5; Handler, A. M., McCombs, S. D., Fraser, M. J. and Saul, S. H. (1998) Proc Natl Acad Sci U S A, 95, 7520-5) of 4% and 8% for the mosquito Aedes aegypti with mariner and Hermes respectively (Coates, C. J., Jasinskiene, N., Miyashiro, L. and James, A. A. (1998) Proc Natl Acad Sci U S A, 95, 3748-51; Jasinskiene, N., Coates, C. J., Benedict, M. Q., Comel, A. J., Rafferty, C. S., James, A. A. and Collins, F. H. (1998) Proc Natl Acad Sci U S A, 95, 3743-7), and 2% for the silkworm Bombyx mori with piggyBac (Toshiki, T., Chantal, T., Corinne, R., Toshio, K., Eappen, A., Mari, K., Natuo, K., Jean-Luc, T., Bernard, M., Gerard, C., Paul, S., Malcolm, F., Jean-Claude, P. and Pierre, C. (2000) Nat Biotechnol, 18, 814).

A transgenic organism for use in the present invention is preferably a multicellular eukaryotic organism, such as an animal, a plant or a fungus. Animals include animals of the phyla cnidaria, ctenophora, platyhelminthes, nematoda, annelida, mollusca, chelicerata, uniramia, crustacea and chordata. Uniramians include the subphylum hexapoda that includes insects such as the winged insects. Chordates includes vertebrate groups such as mammals, birds, reptiles and amphibians. Particular examples of mammals include non-human primates, cats, dogs, ungulates such as cows, goats, pigs, sheep and horses and rodents such as mice, rats, gerbils and hamsters.

Plants include the seed-bearing plants angiosperms and conifers. Angiosperms include dicotyledons and monocotyledons. Examples of dicotyledonous plants include tobacco, (Nicotiana plumbaginifolia and Nicotiana tabacum), arabidopsis (Arabidopsis thaliana), Aspergillus niger, Brassica napus, Brassica nigra, Datura innoxia, Vicia narbonensis, Vicia faba, pea (Pisum sativum), cauliflower, carnation and lentil (Lens culinaris). Examples of monocotyledonous plants include cereals such as wheat, barley, oats and maize.

Techniques for producing transgenic animals are well known in the art. A useful general textbook on this subject is Houdebine, Transgenic animals—Generation and Use (Harwood Academic, 1997)—an extensive review of the techniques used to generate transgenic animals from fish to mice and cows.

Techniques for producing transgenic plants are also well known in the art. Typically, either whole plants, cells or protoplasts may be transfected with a suitable nucleic acid construct encoding a binding domain or binding partner. There are many methods for introducing transforming DNA constructs into cells, but not all are suitable for delivering DNA to plant cells. Suitable methods include Agrobacterium infection (see, among others, Turpen et al., 1993, J. Virol. Methods, 42: 227-239) or direct delivery of DNA such as, for example, by PEG-mediated or liposome-mediated transformation, by electroporation or by acceleration of DNA coated particles. Acceleration methods are generally preferred and include, for example, microprojectile bombardment.

Viral Vectors

The viral vector may be a retroviral vector, and may be derived from or may be derivable from any suitable retrovirus. A large number of different retroviruses have been identified. Examples include: murine leukaemia virus (MLV), human immunodeficiency virus (HIV), simian immunodeficiency virus, human T-cell leukaemia virus (HTLV). equine infectious anaemia virus (EIAV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), Avian myelocytomatosis virus-29 (MC29), and Avian erythroblastosis virus (AEV). A detailed list of retroviruses may be found in Coffin et al., 1997, “retroviruses”, Cold Spring Harbour Laboratory Press Eds: J M Coffin, S M Hughes, H E Varmus pp 758-763.

Details on the genomic structure of some retroviruses may be found in the art. By way of example, details on HIV and Mo-MLV may be found from the NCBI GenBank (Genome Accession Nos. AF033819 and AF033811, respectively).

Retroviruses may be broadly divided into two categories: namely, “simple” and “complex”. Retroviruses may even be further divided into seven groups. Five of these groups represent retroviruses with oncogenic potential. The remaining two groups are the lentiviruses and the spumaviruses. A review of these retroviruses is presented in Coffin et al., 1997 (ibid).

Host range and tissue tropism varies between different retroviruses. In some cases, this specificity may restrict the transduction potential of a recombinant retroviral vector. For this r ason, many gene therapy experiments have used MLV. A particular MLV that has an envelope protein called 4070A is known as an amphotropic virus, and this can also infect human cells because its envelope protein “docks” with a phosphate transport protein that is conserved between man and mouse. This transporter is ubiquitous and so these viruses are capable of infecting many cell types.

Replication-defective retroviral vectors are typically propagated, for example to prepare suitable titres of the retroviral vector for subsequent transduction, by using a combination of a packaging or helper cell line and the recombinant vector. That is to say, that the three packaging proteins can be provided in trans.

A “packaging cell line” contains one or more of the retroviral gag, pol and env genes. The packaging cell line produces the proteins required for packaging retroviral DNA but it cannot bring about encapsidation due to the lack of a psi region. The helper proteins can package a psi-positive recombinant vector to produce the recombinant virus stock. This virus stock can be used to transduce cells to introduce the vector into the genome of the target cells. A summary of the available packaging lines is presented in Coffin et al., 1997 (ibid).

The lentivirus group can be divided into “primate” and “non-primate” lentiviruses. Examples of primate lentiviruses include human immunodeficiency virus (HIV), the causative agent of human auto-immunodeficiency syndrome (AIDS), and simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV). See, for example, Rovira et al., Blood. 2000;96:41114117; Reiser et al., J Virol. November 2000;74(22):10589; Lai et al., Proc Natl Acad Sci U S A October 2000 10;97(21):11297-302; and Saulnier et al., J Gene Med September-October 2000;2(5):317-25.

A distinction between the lentivirus family and other types of retroviruses is that lentiviruses have the capability to infect both dividing and non-dividing cells. In contrast, other retroviruses—such as MLV—are unable to infect non-dividing cells such as those that make up, for example, muscle, brain, lung and liver tissue. Thus, lentiviral vectors may advantageously be used In the present invention since lentiviruses are capable of infecting a wide rang of non-dividing cells, by contrast to certain other retroviruses that requir cell division for stable integration.

A number of vectors have been developed based on various members of the lentivirus sub-family of the retroviridae and a number of these are the subject of patent applications (WO-A-98/18815; WO-A-97/12622). Preferred lentiviral vectors are based on HIV, SIV or EIAV. The simplest vectors constructed from HIV-1 have the complete HIV genome except for a deletion of part of the env coding region or replacement of the nef coding region. Notably these vectors express gag/pol and all of the accessory genes hence require only an envelope to produce infectious virus particles. Of the accessory genes vif, vpr, vpu and nef are non-essential.

One preferred general format for HIV-based lentiviral vectors is, HIV 5′LTR and leader, some gag coding region sequences (to supply packaging functions), a reporter cassette, the rev response element (RRE) and the 3′LTR. In these vectors gag/pol, accessory gene products and envelope functions are supplied either from a single plasmid or from two or more co-transfected plasmids, or by co-infection of vector containing cells with HIV.

Adenoviruses

The adenovirus is a double-stranded, linear DNA virus that does not go through an RNA intermediate. There are over 50 different human serotypes of adenovirus divided into 6 subgroups based on the genetic sequence homology all of which exhibit comparable genetic organisation. Human adenovirus group C serotypes 2 and 5 (with 95% sequence homology) are most commonly used in adenoviral vector systems and are normally associated with upper respiratory tract infections in the young.

The adenoviruses/adenoviral vectors of the invention may be of human or animal origin. As regards the adenoviruses of human origin, preferred adenoviruses are those classified in group C, in particular the adenoviruses of type 2 (Ad2), 5 (Ad5), 7 (Ad7) or 12 (Ad12). More preferably, it is an Ad2 or Ad5 adenovirus. Among the various adenoviruses of animal origin, canine adenovirus, mouse adenovirus or an avian adenovirus such as CELO virus (Cotton et al., 1993, J Virol 67:3777-3785) may be used. With respect to animal adenoviruses it is preferred to use adenoviruses of canine origin, and especially the strains of the CAV2 adenoviruses [Manhattan strain or A26/61 (ATCC VR-800) for example]. Other adenoviruses of animal origin include those cited in application WO-A-94/26914 incorporated herein by reference.

Herpes Simplex Viruses

HSV vectors for use in the invention comprising a polynucleotide of the invention may be derived from, for example, HSV1 or HSV2 strains, or derivatives thereof, preferably HSV1. Derivatives include inter-type recombinants containing DNA from HSV1 and HSV2 strains. Derivatives preferably have at least 70% sequence homology to either the HSV1 or HSV2 genomes, more preferably at least 90%, even more preferably 95%.

The use of HSV strains in therapeutic procedures will require the strains to be attenuated so that they cannot establish a lytic cycle. In particular, if HSV vectors are to be used for gene therapy in humans the polynucleotide should preferably be inserted into an essential gene. This is because if a vector virus encounters a wild-type virus transfer of a heterologous gene to the wild-type virus could occur by recombination. However as long as the polynucleotide is inserted into an essential gene this recombinational transfer would also delete the essential gene in the recipient virus and prevent ‘escape’ of the heterologous gene into the replication competent wild-type virus population.

Attenuated strains may be used to produce the HSV strain of the present invention, here given as examples only, including strains that have mutations in either ICP34.5 or ICP27, for example strain 1716 (MacLean et al., 1991, J Gen Virol 72: 632-639), strains R3616 and R4009 (Chou and Roizman, 1992, PNAS 89: 3266-3270) and R930 (Chou et al., 1994, J. Virol 68: 8304-8311) all of which have mutations in ICP34.5, and d27-1 (Rice and Knipe, 1990, J. Virol 64: 1704-1715) which has a deletion in ICP27. Alternatively strains deleted for ICP4, ICP0, ICP22, ICP6, ICP47, vhs or gH, with an inactivating mutation in VMW65, or with any combination of the above may also be used to produce HSV strains of the invention.

The terminology used in describing the various HSV genes is as found in Coffin and Latchman, 1996. Herpes simplex virus-based vectors. In: Latchman DS (ed). Genetic manipulation of the nervous system. Academic Press: London, pp 99-114. HSV viruses defective in ICP27 are propagated in a cell line expressing ICP27, for example V27 cells (Rice and Knipe, 1990, J. Virol 64: 1704-1715) or 2-2 cells (Smith et al., 1992, Virology 186: 74-86). ICP27-expressing cell lines can be produced by cotransfecting mammalian cells, for example the Vero or BHK cells, with a vector, preferably a plasmid vector, comprising a functional HSV ICP27 gene capable of being expressed in said cells, and a vector, preferably a plasmid vector, encoding a selectable marker, for example neomycin resistance. Clones possessing the selectable marker are then screened further to determine which clones also express functional ICP27, for example on the basis of their ability to support the growth of ICP27—mutant HSV strains, using methods known to those skilled in the art (for example as described in Rice and Knipe, 1990).

Cell lines which do not allow reversion of an ICP27—mutant HSV strain to a strain with functional ICP27 are produced as described above, ensuring that the vector comprising a functional ICP27 gene does not contain sequences that overlap with (i.e. are homologous to) sequences remaining in the ICP27—mutant virus.

Where HSV strains of the invention comprise inactivating modifications in other essential genes, for example ICP4, complementing cell lines will further comprise a functional HSV gene which complements the modified essential gene in the same manner as described for ICP27.

HSV genes may be rendered functionally inactive by several techniques well known in the art. For example, they may be rendered functionally inactive by deletions, substitutions or insertions, preferably by deletion. Deletions may remove portions of the genes or the entire gene. Inserted sequences may include the expression cassette described above.

Mutations are made in the HSV strains by homologous recombination methods well-known to those skilled in the art. For example, HSV genomic DNA is transfected together with a vector, preferably a plasmid vector, comprising the mutated sequence flanked by homologous HSV sequences. The mutated sequence may comprise deletions, insertions or substitutions, all of which may be constructed by routine techniques. Insertions may include selectable marker genes, for example lacZ, for screening recombinant viruses by, for example, β-galactosidase activity.

Mutations may also be made in other HSV genes, for example genes such as ICP0, ICP4, ICP6, ICP22, ICP47, VMW65, gH or vhs. In the case of the VMW65 gene, the entire gene is not deleted since it encodes an essential structural protein, but a small inactivating insertion is made which abolishes the ability of VMW65 to transcriptionally activate IE genes (Ace et al., 1989, J Virol 63: 2260-2269).

Baculovirus

Baculovirus vectors may moreover be employed in the invention. The baculovirus Autographa californica multiple nuclear polyhedrosis virus (AcMNPV) is a DNA virus which can be replicate only in cells of certain lepidopteran insects and has been used widely for expression of recombinant proteins in insect cells. Baculoviruses such as AcMNPV have been used recently for introducing heterologous DNA with high efficiency in a variety of mammalian cells, such as a hepatoma cell line and primary liver cells, and endothelial cells (Boyce F M, Bucher N L (1996) Baculovirus-mediated gene transfer into mammalian cells. Proc Natl Acad Sci U S A 93, 2348-52; Airenne K J, Hiltunen M O, Turunen M P, Turunen A M, Laitinen O H, Kulomaa M S, Yla-Herttuala S (2000) Baculovirus-mediated periadventitial gene transfer to rabbit carotid artery. Gene Ther 7,1499-1504). Moreover, baculovirus vectors for gene transfer, methods for introducing heterologous DNA into their genome and procedures for recombinant virus production in insect cell cultures are available commercially; furthermore, baculoviruses cannot normally replicate in mammalian cells, so there is no need to engineer them for this use.

Vector Construction

Construction of vectors according to the invention may employ conventional ligation techniques. Isolated viral vectors, plasmids or DNA fragments are cleaved, tailored, and religated in the form desired to generate the plasmids required. If desired, analysis to confirm correct sequences in the constructed vectors is performed in a known fashion. Transposon presence and/or mobilisation may be measured in a cell directly, for example, by conventional Southern blotting, dot blotting, PCR or in situ hybridisation, using an appropriately labelled probe which may be based on a sequence present in the transposon. Those skilled in the art will readily envisage how these methods may be modified, if desired.

Marker Genes

Vectors useful in the present invention are advantageously provided with marker genes to facilitate transposon identification and localisation. Preferred marker genes include genes which encode fluorescent polypeptides. For example, green fluorescent proteins (“GFPs”) of cnidarians, which act as their energy-transfer acceptors in bioluminescence, can be used in the invention. A green fluorescent protein, as used herein, is a protein that fluoresces green light, and a blue fluorescent protein is a protein that fluoresces blue light. GFPs have been isolated from the Pacific Northwest jellyfish, Aequorea victoria, from the sea pansy, Renilla reniformis, and from Phialidium gregarium. (Ward et al., 1982, Photochem. Photobiol., 35: 803-808; Levine et al., 1982, Comp. Biochem. Physiol.,72B: 77-85). See also Matz, et al., 1999, ibid for fluorescent proteins isolated recently from Anthoza species (accession nos. AF168419, AF168420, AF168421, AF168422, AF168423 and AF168424).

A variety of Aequorea-related GFPs having useful excitation and emission spectra have been engineered by modifying the amino acid sequence of a naturally occurring GFP from Aequorea victoria (Prasher et al., 1992, Gene, 111: 229-233; Heim et al., 1994, Proc. Natl. Acad. Sci. U.S.A., 91: 12501-12504; PCT/US95/14692). As used herein, a fluorescent protein is an Aequorea-related fluorescent protein if any contiguous sequence of 150 amino acids of the fluorescent protein has at least 85% sequence identity with an amino acid sequence, either contiguous or non-contiguous, from the wild-type Aequorea green fluorescent protein (SwissProt Accession No. P42212). More preferably, a fluorescent protein is an Aequorea-related fluorescent protein if any contiguous sequence of 200 amino acids of the fluorescent protein has at least 95% sequence identity with an amino acid sequence, either contiguous or non-contiguous, from the wild type Aequorea green fluorescent protein of SwissProt Accession No. P42212. Similarly, the fluorescent protein may be related to Renilla or Phialidium wild-type fluorescent proteins using the same standards.

Aequorea-related fluorescent proteins include, for example, wild-type (native) Aequorea victoria GFP, whose nucleotide and deduced amino acid sequences are presented in GenBank Accession Nos. L29345, M62654, M62653 and others Aequorea-related engineered versions of Green Fluorescent Protein, of which some are listed above. Several of these, i.e., P4, P4-3, W7 and W2 fluoresce at a distinctly shorter wavelength than wild type.

### USES OF THE INVENTION

In a highly preferred embodiment, the present invention is particularly useful in enabling the induction of transposition in whole organisms by introducing the transposon into target cells using a viral system followed by induction of transposition using constitutively expressed or inducible transposase systems. Transposase may be expressed in all tissues. Alternatively, transposase may be expressed in a tissue specific manner, by the use of, for example, tissue specific chromatin opening domains. In this way the tissues in which transposition is induced may be controlled.

Transposons, and sites from which transposons have been excised, may be identified by sequence analysis. For example, Minos typically integrates at a TA base pair, and on excision leaves behind a duplication of the target TA sequence, flanking the four terminal nucleotides of the transposon. The presence of this sequence, or related sequences, may be detected by techniques such as sequencing, PCR and/or hybridisation.

Inserted transposons may be identified by similar techniques, for example using PCR primers complementary to the terminal repeat sequences.

The invention allows functional mapping of a genome by permitting precise gene modulation and subsequent detection using transposons.

The invention, in an advantageous embodiment, allows genes to be ablated by transposon insertion and then specifically identified through the transposon “tag” without requiring costly and time-consuming genetic analyses, and frequently without significant amounts of sequencing. It is a particular advantage of the invention that both alleles of a gene may be inactivated; the transposon advantageously contains an inducible promoter (for example, the tet inducible system) 5′ to the splice acceptor, which is induced to make an antisense transcript of the gene in question. The antisense RNA inactivate the RNA from the intact allele resulting in a complete or partial knock out of both alleles of the gene.

Upregulation of a gene is achieved by introducing a strong transcriptional enhancer 3′to an internal ribosome binding site coupled to the reporter (such as GFP). Different enhancers would be used for different cell types, for example an immunoglobulin enhancer for B cells). The integration of a transposon at the 3′ end of a gene would result in a mRNA which also translates the reporter via the internal ribozyme binding site and upregulate the gene through the enhancer (or LCR type sequence).

The two methods can also be combined by including the reverse promoter and a splice acceptor 5′ to the IRES. Both knockouts and upregulators would be present in the same library.

All insertions would take place in cell lines that already contain the tet-induction system and an inducible transposase.

In an alternative embodiment, the invention uses transposons to “mark” genes whose expression is modulated by external stimuli. Thus, a cell line which has been exposed to transposon mobilisation with a marked transposon is subjected to treatment with an external stimulus, such as a candidate drug or other test agent, and modulation of the expression of the marker observed. Cells in which the marker is over or under-expressed are likely to have the transposon inserted in or near a gene which is upregulated or downregulated in response to the stimulus. The invention may thus be used to provide in vivo enhancer trap and exon trap functions, by inserting transposons which comprise marker genes which are modulated in their expression levels by the proximity with enhancers or exons. Such applications are described in general in EP 0955364 and known in the art.

This approach is useful for the study of gene modulation by drugs in drug discovery approaches, toxicology studies and the like. Moreover, it is applicable to study of gene modulation in response to natural stimuli, such as hormones, cytokines and growth factors, and the identification of novel targets for molecular intervention, including targets for disease therapy in humans, plants or animals, development of insecticides, herbicides, antifungal agents and antibacterial agents.

The approaches set forth above may be applied in different cell lines, derived from different organisms or different tissues, in order to monitor differential effects of the stimuli under study.

The invention is further described in the following examples, which are intended to be illustrative and non-limiting.

### EXAMPLES

Examples 1 and 2 describe the use of baculovirus for high efficiency introduction of transposons into cells expressing transposase using pMiLRgeo and pMiLRneo respectively. Examples 3 and 4 describes the use of retrovirus vectors for introduction of transposons into cells expressing transposase.

### Example 1

Use of Baculovirus for High Efficiency Introduction of Transposons into Cells Expressing Transposase

The Autographa califomica nuclear polyhedrosis virus (AcNPV) is the most commonly used virus for expression of heterologous proteins. Vectors are available commercially, for example from Clontech, from whom detailed descriptions thereof may be obtained.

A brief description of the construction of a recombinant AcNPV comprising a transposon comprising an exon trap is given below:

A. Transposon.

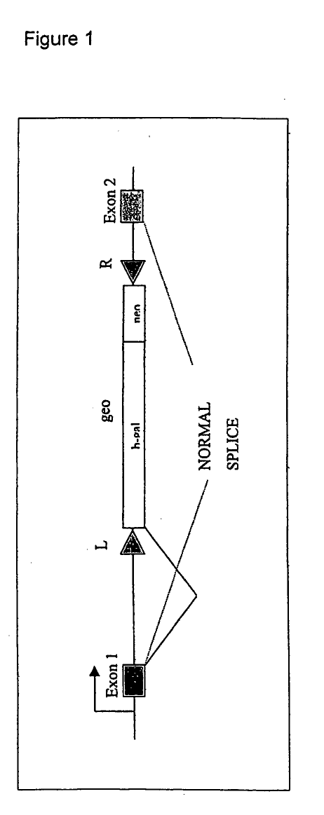

The transposon is Minos exon trap vector pMiLRgeo, described in Klinakis et al. 2000 EMBO Reports 1: 416-421. MiLRgeo comprises a gene trap construct, consisting of a splice acceptor site followed by an in-frame fusion of the E. coli beta-galactoside gene with a prokaryotic gene conferring resistance to the antibiotic neomycin (a gene trap fusion referred as geo, Scarnes et al., 1995, Proc. Natl Acad. Sci. USA, 92, 6592-6596). The geo gene does not contain a translational initiation signal, and expression of the beta-gal and neo phenotypes is dependent upon the insertion of the vector in an intron in the correct orientation, so that splicing generates a fusion mRNA that has necessary translational initiation. The exon trap vector, inserted into an intron, is shown schematically in FIG. 1.

The exon trap can include any of several reporter genes that are available, such as GFP, available from Clontech, beta-galactosidase, beta-lactamase, beta-glucuronidase and luciferase. It may also include selectable genes, such as genes conferring resistance to neomycin, puromycin and hygromycin. Alternatively, it may comprise a fusion between a reporter and a selectable marker, such as geo that is used in this example.

B. Construction of Recombinant Virus.

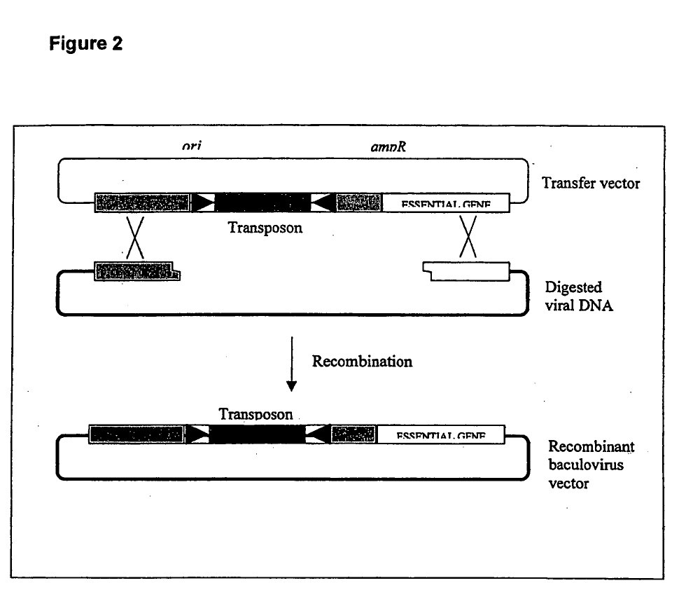

Procedures for engineering recombinant AcNPV are described and all components are available commercially, as indicated above, for example from Clontech. The procedure is briefly the following: The viral genome is a 134 kbp long double-stranded DNA circle, and direct manipulation of it is difficult. Recombinant baculovirus vectors are, therefore, constructed in two steps. First, the target sequence (in this case the entire transposon, in the form of a restriction fragment) is cloned into a modified viral gene contained in a small plasmid vector (transfer vector). The gene is the polyhedrin gene of which the coding sequence has been deleted and replaced with a multiple cloning site (MCS). The transfer vector contains an antibiotic resistance gene and an origin of replication, so that it can be propagated in E.coli cells, but not in insect or other eukaryotic cells. The transposon is cloned between the promoter and the polyadenylation signal of the polyhedrin virus. In a second step, the transfer vector carrying the transposon is cotransfected into appropriate insect cells (e.g. cell line SF9 from the lepidopteran Spodoptera frugiperda) along with a viral vector. Double recombination between the transfer vector and the viral vector results in a recombinant virus containing the transposon (FIG. 2).

In the specific example given above, the transfer vector contains a complete version of an essential viral gene downstream from the MCS. The viral vector is engineered so that the essential gene is mutated. Double recombination restores function of this gene and provides a strong genetic selection for recombinant virus.

C. Generation of Cells Inducibly Expressing Transposase.

Transient expression of transposase is required to mobilise a transposon from its original position on the viral DNA to new chromosomal positions. Continued expression of transposase after integration of the transposon is undesirable because it will lead to re-mobilisation of the transposon. To achieve regulatable expression of transposase in cells, a two-transgene scheme will be employed: Cells are stably transgenic with two constructs: one containing the transposase gene under the control of an activatable promoter and a second containing a stably expressed gene encoding the inducible transcriptional activator of said promoter. A widely used system of this kind in mammalian cells is the tetO promoter-operator, combined with the tetracycline/doxycycline-repressible transcriptional activator tTA, also called Tet-Off gene expression system (Gossen, M. & Bujard, H. (1992) Tight control of gene expression in mammalian cells by tetracycline responsive promoters. Proc. Natl. Acad. Sci. USA 89:5547-5551), or the doxycycline-inducible rtTA transcriptional activator, also called Tet-On system (Gossen, M., Freundlieb, S., Bender, G., Muller, G., Hillen, W. & Bujard, H. (1995) Transcriptional activation by tetracycline in mammalian cells. Science 268:1766-1769).

In the Tet-Off system, gene expression is turned on when tetracycline (Tc) or doxycycline (Dox; a Tc derivative) is removed from the culture medium. In contrast, expression is turned on in the Tet-On system by the addition of Dox. Procedures for establishing cell lines carrying the transcriptional activator gene and the Tet-regulatable gene stably integrated in its chromosomes have been described. For example see http://www.clontech.corn/techinfo/manuals/PDF/PT3001-1.pdf. In the specific example, the Tet-On system is employed for tetracycline-inducible expression of Minos transposase in a mammalian cell line. A doubly transgenic line is generated by standard illegitimate recombination technology. Two constructs are used: First, a construct containing the rtTA gene under a constitutive promoter expressed in the target cells. An example of such construct is the pTet-On plasmid (Clontech) which contains the gene encoding the rtTA activator under control of the Cytomegalovirus immediate early (CMV) promoter. The rtTA transcriptional activator encoded by this construct is active only in the presence of Doxycycline. The second construct contains the Minos transposase gene under control of the tetracycline-response element, or TRE. The TRE consists of seven direct repeats of a 42-bp sequence containing the tet operator (tetO), and is located just upstream of the minimal CMV promoter, which lacks the enhancer elements normally associated with the CMV immediate early promoter. Because these enhancer elements are missing, there is no “leaky” expression of transposase from the TRE in the absence of binding by rtTA. An example of such construct is the pTRE2 plasmid (Clontech) in the MCS of which is inserted the gene encoding Minos transposase. In cells stably transformed with the two constructs, rtTA is expressed but does not activate transcription of Minos transposase unless Doxycycline (0.1-1 micrograms/ml) is added in the medium.

D. High Fficiency Generation of Random Insertions of the Transposon Int Chromosomes.

Generation of insertions is accomplished in three steps: First, transposon-loaded recombinant baculovirus is used to infect the doubly transgenic cells at high titres, to achieve infection of individual cells by multiple copies of the virus. The virus cannot replicate in mammalian cells, but its DNA moves into the nucleus and has been shown to be accessible to the transcriptional apparatus. As a second step, cells are exposed to appropriate concentrations of Doxycycline to induce expression of transposase. In a third step, cells are moved to a medium without doxycycline to arrest transposase expression, and supplemented with appropriate antibiotic (G418 for this example) to select for cells that have the transposon inserted. In this specific example, G418 selection will select only cells that contain an “active” exon trap, i.e. the transposon has inserted into an intron of an expressed gene in such a way that an active neo protein is expressed as result of splicing.

### Example 2

Use of Baculoviruses to Introduce Transposons into Eukaryotic Cells

Summary

A. The use of transposable elements as genome-wide insertional mutagenesis agents can be limited by low transfection rates of DNA into cells. One possible way to overcome this obstacle is to use a high-infectivity virus as a vehicle to introduce transposons into cells. We have tested the ability of a Minos transposon to transpose, in the presence of cognate transposase, from recombinant baculovirus carrying the transposon, into chromosomes of infected mammalian cells. Recombinant Autographa californica nuclear polyhedrosis virus (AcNPV) was constructed containing a Minos transposon carrying an antibiotic resistance marker gene. Recombinant virus was used to infect a cell line in the presence and absence of transposase and numbers of stably transformed colonies were determined after selection with antibiotic. The presence of transposase resulted in 200-400fold stimulation of stable integration of the transposon. Southern arialysis showed that individual colonies carried 1-7 copies of the transposon integrated by transposase-mediated events. In a separate line of experiments, infectivity of baculovirus was tested in a number of mammalian cell lines using a recombinant AcNPV expressing Green Fluorescent Protein (GFP). Between 20% and 80% of cells expressed GFP after infection with this virus. It is concluded that recombinant baculovirus can be used as an efficient vehicle to introduce transposons into mammalian cells.

B. Transgenic mice and cell lines inducibly expressing transposase are useful for inducing transposition of cognate transposons that are already integrated in chromosomes or that reside on episomal DNA, such as recombinant baculovirus DNA. Transgenic mice were generated carrying a construct comprising the Minos transposase under the control of the tet operator (Gossen, M. & Bujard, H. (1992) Proc. Natl. Acad. Sci. USA 89:5547-5551). Expression of transposase can be regulated by doxycycline in double trangenic mice carrying this construct and a gene encoding the tTA or rtTA transcriptional activators (Gossen, M., Freundlieb, S., Bender, G., Muller, G., Hillen, W. & Bujard, H. (1995) Science 268:1766-1769). Double transgenics can be used directly for in vivo transposition experiments, of for generation of primary or immortalized cell lines (Cascio SM. (2001) Artif Organs. 25: 529-538).

C. Cell lines inducibly expressing transposase can be generated by stable integration of two constructs: A construct encoding an inducible transcriptional activator (such as the tet activator rtTA) and a construct encoding transposase under the control of the tet operator (tetO). Plasmids containing these constructs were tested for tetracycline-regulatable expression of transposase by co-transfecion into HeLA cells with a plasmid containing a Minos transposon. Treatment with doxycycline increased transposon integration rates 26-fold over non-treated controls. The constructs encoding tet activator and tetO-controlled transposase are used to generate cell lines with stable chromosomal insertions, which can produce transposase inducibly.

Materials and Methods

Recombinant baculovirus and olasmids: Recombinant AcNPV baculoviruses were generated using the BacPac Baculovirus Expression System (Clontech) according to manufacturer's instructions (http://www.clontech.com/techinfo/manuals/PDF/PT1260-1.pdf). Recombinant plasmids were constructed by standard methodologies.

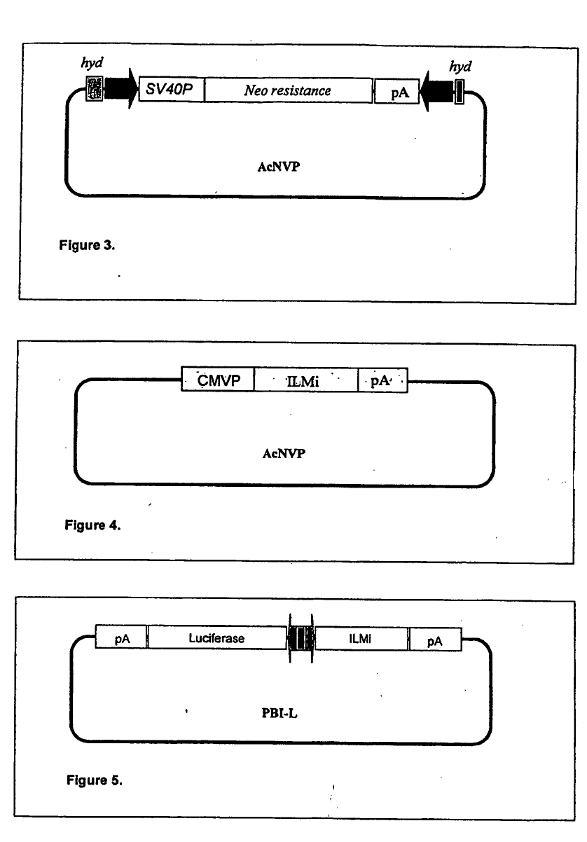

BacMiLRneo (FIG. 3) is a recombinant AcNPV virus containing a transposon that consists of the Minos inverted repeats (block arrows) flanking the neo gene under the control of the early SV40 promoter. A Minos transposon containing the SV40neo gene was subcloned as a KpnI-SacI fragment from plasmid pMiLRneo (Klinakis, A. G., L. Zagoraiou, D. K. Vassilatis and C. Savakis (2000). EMBO Reports. 1: 416-421) into the respective sites of the pBacPAK9cloning vector (Clontech). The resulting plasmid (pBacPAK9MiLRneo) was used for generation of recombinant AcNPV virus by recombination in SF21 cells, according to manufacturer's instructions (http://www.clontech.com/techinfo/manuals/PDF/PT1260-1.pdf).

BacCMV/ILMi (FIG. 4) is a recombinant AcNPV virus containing the processed (intronless) gene encoding Minos transposase under the control of the CMV early promoter. CMV/ILMi was subcloned as a Pvull-Nrul fragment from plasmid pCMV/ILMi (Zagoraiou L., D. Drabek, S, Alexaki, J. A. Guy, A. G. Klinakis, A. Langeveld, G. Skavdis, C. Mamalaki, F. Grosveld and C. Savakis (2001) Proc. Natl. Acad. Sci. USA 98: 11474-11478) into the Smal site of the pBacPAK9 cloning vector (Clontech). The resulting plasmid (pBacPAK9CMV/ILMi) was used for generation of recombinant AcNPV virus by recombination in SF21 cells, according to manufacturer's instructions.

pPBI-L/ILMi (FIG. 5) is a helper plasmid based on the pBI-L cloning vector (Clontech). It contains a bidirectional promoter consisting of two copies of a minimal CMV promoter flanking a Tetracycline Response Element (TRE). The TRE consists of 7 direct repeats of a 42 bp sequence containing the tet operator (tetO). Plasmid pPBI-L/ILMi was constructed by cloning the intronless transposase gene from plasmid pHSS6hsILMi20 (Klinakis, A. G., T. G. Loukeris, A. Paviopoulos and C. Savakis (2000) Insect Mol. Biol. 9:269-275) as a Clal-Sall fragment into the respective sites of pBI-L.

Infection of cultured cells with baculovirus and selection of stably transfected cells: HepG2 cells were seeded on six-well plates (750000 cells per well) containing 2 ml of medium (DMEM, Gibco BRL). The medium was replaced one day later and baculovirus was added in a small volume of PBS. In superinfection experiments the second virus was added 4 hours after the first. The virus was removed 15 hours post infection by replacing the medium. The cells were trypsinised 48 hours post infection and ½ and 1/20 of them were seeded on 60 mm plates. Treatment with G418 started 72 h post infection. After 25 days, the neo resistant colonies were isolated and propagated in G418 containing medium.

Transfection of cultured cells with plasmid DNA and selection of stably transfected cells: Transfection of HeLa cells and selection for stably transfected G418 resistant cells was performed as described previously (Klinakis, A. G., L. Zagoraiou, D. K. Vassilatis and C. Savakis (2000) EMBO Reports. 1: 416421).

Results

1. Infection of Mammalian Cells with Baculovirus.

To determine efficiencies of infection of mammalian cells by baculovirus, a recombinant baculovirus that carries a green fluorescent protein cassette (van Loo N D, Fortunati E, Ehlert E, Rabelink M, Grosveld F, Scholte BJ. (2001) J Virol. 75: 961-970) was used to infect several cell lines at a multiplicity of infection (MOI) of 200. Infection efficiencies varied between approximately 20% in the human breast cancer lines MCF7 and T47D, 50% in the human hepatoma HepG2 line and 80% in the rat embryonic fibroblast Ref1 line.

2. Transposition of Minos from a Recombinant Baculovirus.

To test whether a Minos transposon carried by a recombinant baculovirus can be mobilized by transposase to insert into cell chromosomes, human hepatoma HepG2 cells were infected with recombinant BacMiLRneo virus with or without recombinant helper BacCMV/ILMi virus and stably transfected colonies were recovered after selection with G418. As shown in Table 1, coinfection or super-infection of the transposon-carrying virus with the helper plasmid increased formation of resistant colonies by approximately 200-400 fold relative to infection with the transposon-carrying virus alone.

TABLE 1
Stable integration of transposon from recombinant baculovirus
into HepG2 chromosomes

| | | Resistant |
|---|---|---|
| BacMiLRneo MOI | BacCMV/ILMi MOI | colonies |
| 50 | 0 | 0 |
| 250 | 0 | 8 |
| 250 | 250 | 2080 |
| | co-infection | |
| 250 | 250 | 3400 |
| | super-infection | |
| 50 | 500 | 1820 |
| | super-infection | |
| 500 | 50 | 1960 |
| | super-infection | |

To determine the numbers and nature of stable insertions, twelve G418 resistant colonies were propagated and subjected to Southern analysis, using appropriate probes and restriction enzymes that do not cut within the transposon. The banding patterns showed that individual colonies contained between 1 and 7 insertions of the transposon at different positions of the genome. Restriction patterns did not show presence of vector DNA flanking the transposon.

3. Induction of Transposase Expression by Doxvcyclin in a Cell Line.

To determine inducibilty of transposase expression, HeLa cells were co-transfected with three plasmids:

pPBI-L/ILMi, expressing luciferase and Minos transposase under control of a bi-directional tetO operator

prtTAM2 (Clontech) containing a rtTA activator expression cassette (the rtTA activator is inactive in the absence of inducible by tetracyclin or doxycyclin)

pMiLRneo, containing a Minos transposon with a neo resistance cassette (Klinakis, A. G., L. Zagoraiou, D. K. Vassilatis and C. Savakis (2000) EMBO Reports. 1: 416421)

and subjected to selection with G418. As shown in Table 2, treatment with doxycyclin resulted in 18-fold increase of transposon integration relative to untreated controls.

TABLE 2
Induction of transposase expression by doxyciclin

| | −dox | +dox | −dox | +dox |
|---|---|---|---|---|
| pBI-L | — | — | 1 μg | 1 μg |
| pBI-L/ILMi | 1 μg | 1 μg | | |
| prtTAM2 | 1 μg | 1 μg | 1 μg | 1 μg |
| pMiLRneo | 2 μg | 2 μg | 2 μg | 2 μg |
| Number of colonies | 330 | 6000 | 100 | 160 |

Stably transfected cell lines carrying the tet activator expression cassette from plasmid prtTAM2 and the tetO transposase/luciferace cassette from plasmid pBIL/ILMi is generated by standard procedures. To generate high frequency transposition, a stable cell line expressing tet inducible transposase is infected with recombinant baculovirus carrying a transposon. Transposase is induced transiently by treatment with doxycyclin and catalyzes transpositions from the episomal viral DNA into chromosomes. Removal of inducer results in removal of transposase and stabilizes insertions of the transposons. Cells carrying stable integrations of the transposon are then selected. Selection is based on selectable or screenable markers carried by the transposon (e.g. antibiotic resistance or expression of autofluorescent proteins).

4. Generation of Transgenic Mice Inducible Expressing Transposase

Three trangenic lines were generated by standard procedures carrying the the tetO transposase/luciferase cassette from plasmid pBIL/ILMi. Double transgenics that inducibly express transposase can be generated by crossing these mice with transgenics carrying a rtTA (or a tTA) expression cassette.

### Example 3

Use of Retrovirus Vectors for Introduction of Transposons into Cells Expressing Transposase

Construction of Retroviral Vectors

The transposon is cloned into a retroviral/lentiviral vector by standard recombinant DNA techniques (see e.g. Hoeflich et al 2000, nature 406, p82, where a β globin cassette is exchanged for a lacZ cassette in an existing retroviral vector). The recombinant transposon/viral vector plasmid DNA is isolated by standard procedures and transfected into the viral packaging cell line as described (Hoeflich et al., 2000). Virions are collected and concentrated as described by Gallardo et al., 1997 (Blood, 90, 952-57). Target cells are infected in the presence of polybrene (8 μg/ml) as described by Sadelain et al., (PNAS 92, 6728-32, 1995) to establish the starting population of cells containing a transposon insertion after viral integration.

The starting population of target cells for infection are either established cell lines, primary cell cultures (from mouse or human or other animals; e.g.. Methods in Enzymology Vol 58, Cell Culture (1979), Academic press Inc. San Diego. Editors W. B. Jakoby and I. H. Pastan, Editors in chief: S. P. Colowick and N. O.) or immortalised cells (e.g. Jat P S, Noble M D, Ataliotis P, Tanaka Y, Yannoutsos N, Larsen L, Kioussis D. Direct derivation of conditionally immortal cell lines from an H-2Kb-tsA58 transgenic mouse. Proc Natl Acad Sci U S A. 1991; 88(12): 5096-100) or embryonic stem cells. Each of these target cells is first transfected with (or infected with a retrovirus containing) a construct containing a Tet inducible (Baron U., Nucl Acid Res., 25, 2723-29, 1997 and r ferences cited therein), or tamoxifen inducible transposase [a modified oestrogen receptor domain (Indra et al., Nucl Acid Res. 27, 4324-27, 1999) coupled to the transposase which retains it in the cytoplasm until tamoxifen is given to the culture], or a RU418 inducible transposase (operating under the same principle as with the glucocorticoid receptor; see Tsujita et al., J. Neuroscience, 19, 10318-23, 1999). The gene for the transposase in introduced by standard transfection methods or is present in the transgenic animal from which the primary cells are isolated.

The transposase gene is introduced into those animals either via microinjection of fertilised eggs by standard procedures (Manipulating the mouse embryo, Hogan et al., Cold Spring Harbor Press, 1994) or introduced into embryonic stem cells via homologous or non homologous recombination. The ES cells are injected into blastocysts to obtain transgenic animals via standard procedures (Manipulating the mouse embryo, Hogan et al., Cold Spring Harbor Press, 1994).

The transposon construct is introduced into mice via microinjection of fertilised eggs (Manipulating the mouse embryo, Hogan et al., Cold Spring Harbor Press, 1994) or embryonic stem cells (Manipulating the mouse embryo, Hogan et al., Cold Spring Harbor Press, 1994). The transposon is made to jump in mouse somatic tissues to either isolate somatic cells with different transposon integration or/and germ line tissue (preferably sperm) to establish a population of mice in the next generation that contain the transposon in different positions.

An alternative in the animal population is to introduce the transposon via a retroviral infection step (using lentivirus or retrovirus vectors, as above) which establishes a starting population of different germ line insertions. Inducing transposase in the infected germ cells will increase the population of transposons, which is spread by breeding.

Transposition is monitored by northern blot analysis, PCR or FISH.

### Example 4

Use of a Lentivirus Vectors for Introduction of Transposons into Cells Expressing Transposase

A Plasmids

PBO-MG1

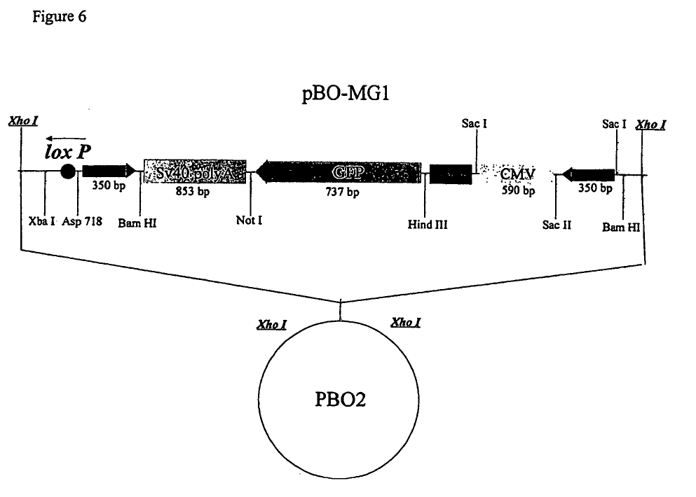

The construct pBO-MG1 (FIG. 6) is a self-inactivating lentiviral vector plasmid containing Minos transposon sequences flanking the GFP gene driven by the CMV promoter. Plasmid pBO-MG1 was obtained by subcloning the Xho I insert fragment of the plasmid pMiCMVGFP, into the Xho I site of the plasmid pBO2. The parent plasmid pBO2 was derived from the plasmid CS-CG (Myoshi et al. (1998) J. Virol 72, 8150-57).

Transposon MiCMVGFP is constructed as follows: The plasmid pMILRTetR (Klinakis et al. (2000) Ins. Mol. Biol. 9, 269-275 (2000b is cut with BamH I and re-ligated to remove the tetracycline resistance gene between the Minos ends, resulting in plasmid pMILRΔBamH1. An Asp7l8/Sacl fragment from pMILRΔBam H1, containing the Minos inverted repeats and original flanking sequences from D. hydei, is cloned into plasmid pPolyIII-I-lox (created by insertion of the loxP oligo:

ATAACTTCGTATAGCATACATTATACGAAGTTAT

into the Asp718 site of the vector pPolyIII-I (accession No. M18131 ), resulting in plasmid ppolyMILRΔBamH. The final construct (pMiCMVGFP, FIG. 1) used for the generation of transgenic mice, is created by inserting into the Spe I site of ppolyMILRΔBamH1 the 2.2 kb SpeI fragment from plasmid pBluescriptGFP, containing a humanised GFP gene (from Clontech plasmid pHGFP-S65T) driven by the CMV promoter and followed by the SV40 intervening sequence and polyadenylation signal.

Plasmid pNT-1

A 1 kb Clal Not I fragment containing Minos transposase cDNA was cloned into Cla INot of Pev3 (Clare Gooding, Biotechnology Dept, Zeneca, Macclesfield, UK). A 3.8 kb ClaI Asp 781 fragment from the resulting plasmid (containing minos transposase cDNA followed by an intron with RNA splicing site and a polyadenylation signal from the human β globin gene) was subcloned into Cla IAsp7l8) of the pBluescript SK (Stratagene, La Jolla, Calif., USA) creating the plasmid pBlue/transposase/3′β. Plasmid pNT-1 was derived from the plasmid pBlue/transposase/3′β by cloning a 580 bp blunt ended Sac I- Spe I fragment, spanning the CMV promoter, into the EcoR V site.

pMDLg/RRE expresses the HIV-1 gag and pol proteins.

pRSV.REV expresses the HIV-1 REV protein.

pMD.G expresses the envelope G-glycoprotein of VSV (Vesicular Stomatitis Virus). Details relating to pMDLg/RRE plasmid, the pRSV.REV plasmid and the pMD.G plasmid can be found in Dull, T., Zufferey, R., Kelly, M., Mandel, R. J., Nguyen, M., Trono, D. and Naldini, L. (1998) J. Virol, 72, 8463-8470.

All plasmids were prepared by using the Qiagen Endotoxin-Free Maxi- or Giga-prep kit.

B Virus Production

The virus production protocol described below was used.

1. Human Embryonal Kidney (HEK) cell line 293 T was used for the packaging of lentiviral vectors (Dull et al. 1998). The packaging line 293T was grown to 70% confluence in 10 mis of DMEM-10% FCS in 20×10cm dishes.

2. The following plasmids were mixed in the amounts shown per 10 cm dish:

| | |
|---|---|
| pBO-MG1 plasmid | 10.0 μg |
| pMDLg/RRE plasmid | 6.5 μg |
| pRSV.REV plasmid | 2.5 μg |
| pMD.G plasmid | 3.5 μg |

3. To the DNA mix 500 μl of freshly diluted 0.25M CaCl₂ was added followed by 500 μl of 2×BBS solution. The mixture was swirled gently and incubated at room temperature for 15 min.

(2×BBS (1 litre ) comprises dissolved NaCl 16.36 g, BES (N,N-bis-(2-Hydroxyethyl)-2aminoethanesulfonic acid) 10.65 g [from Calbiochem #391334], Na₂HPO₄ 0.21 g in 900 ml H₂O, titrated to pH6.95 with 1M NaOH and brought to 1 litre with H2O, filter sterilized and stored frozen at −20° C.)

4. The DNA-CaCl₂ mixture was added dropwise into the dish.

5. Dishes were placed in a 37° C. incubator under 3% CO₂ for 12-16 hrs.

6. The medium was changed and the plates incubated under the same conditions for 24 hrs., following which the medium was collected and the virus particles harvested.

7. This was repeated 2×more at 24 hr. intervals and the medium filtered through 0.45 μm cellulose acetate filter after each harvest.

8. Virus particles were concentrated by spinning in Beckman SW28 rotor at 19.4K rpm for 2 hrs. at room temperature. The pellet was resuspended in 1 ml Hanks Balanced Salt Solution (HBBS) and re-spun in a Beckman SW55 rotor at 21K for 2 hrs. at room temperature.

9. The viral pellet was suspended in 200 μl HBBS and vortexed at low speed for 1-2 hrs at room temperature.

10. The suspension was spun in a microfuge and the supematant stored as 10-50 μl aliquots at −80° C.

C Transduction With pBO-MG1

1) Virus was harvested from the 293T producer cell line and used for a PCR assay to determine viral titres.

2) The target cell line (MEL- Murine Erythroleukemia) was seeded at subconfluent concentrations of 1×10⁵ cells/ml and allowed to grow O/N at 37° C., 5% CO₂.

3) The virus was suitably diluted to give an overall MOI of 50 and added to the medium. Incubation with the virus was O/N under the conditions described above.

4) The cells were harvested and cloned by limiting dilutions. Clones positive for the transgene were expanded and single-copy clones transfected with the plasmid p-NT2 harboring the transposase, driven by the CMV promoter, using the transfection reagent “Superfect” (Qiagen # 301305) and incubated O/N under the above conditions.

5) Cells were harvested 48 hrs. following the introduction of the transposase and genomic DNA prepared for analysis by southern blotting.

6) Genomic DNA was digested with Bspe I, which has a single site in the 3′ LTR and probed with an end DNA fragment probe.

D Results

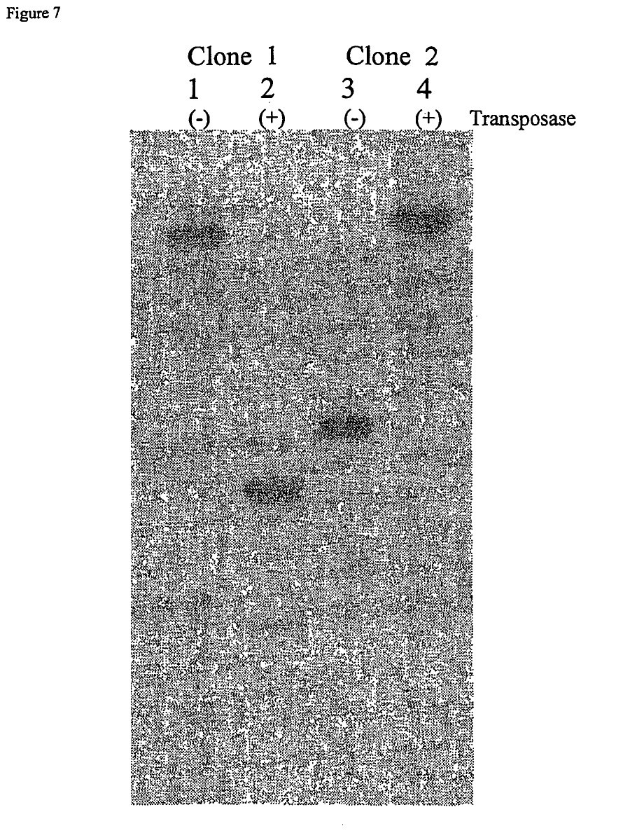

FIG. 7 shows a Southern blot of genomic DNA from clones 1 and 2 of MEL cells carrying an integrated copy of the lenti-Minos-GFP virus. The DNA was digested with BspE I and probed with a 3′LTR end fragment probe. Lanes 2 & 4 have DNA from the clones transfected with the plasmid pNT-1, carrying the CMV driven transposase gene resulting in a transposition that gives a new band that hybridises with end fragment probe.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

1. A method for producing a library of genetic mutations in a cell population by insertional mutagenesis, wherein a viral Vector comprising a transposon is used to deliver said transposon to said cell population, which cell population stably expresses a regulatable cognate transposase for said transposon, and the transposon is mobilised to give rise to the genetic mutations.

2. The method of claim 1 wherein the population of cells is a transgenic non-human animal.

3. The method of claim 1 wherein a nucleic acid encoding the transposase has been delivered to the cell using an integrating expression vector and the cell has been cultured to achieve stable expression of the transposase.

4. The method of claim 1 wherein the expression of the transposase is under the control of an inducible promoter.

5. The method of claim 4 wherein the inducible promoter is selected from a tetracycline-inducible promoter, a promoter from the beta globin locus and an oestrogen-inducible promoter.

6. The method of claim 1 wherein said viral vector is selected from a retroviral, lentiviral, adenoviral or bacculo viral vector.

7. The method of claim 1 wherein the transposon is selected from Minos, mariner, Hermes and Piggybac.

8. The method of claim 1 wherein the cell population which stably expresses the transposase is an established cell line, a primary culture or a stem cell.

9. The method of claim 8 wherein said stem cell is an embryonic stem cell.
